(19) United States
(12) Patent Application Publication
(10) Pub. No.: US 2016/0262399 A1
(43) Pub. Date: Sep. 15, 2016

(54) Bar Compositions Comprising Platelet Zinc Pyrithione

(71) Applicant: The Procter & Gamble Company, Cincinnati, OH (US)

(72) Inventors: Edward Dewey SMITH, III, Mason, OH (US); Jason Edward COOK, Anderson Township, OH (US); Brian Joseph LIMBERG, Milford, OH (US)

(21) Appl. No.: 15/157,555

(22) Filed: May 18, 2016

Related U.S. Application Data
(63) Continuation of application No. 13/036,889, filed on Feb. 28, 2011, now abandoned.

(51) Int. Cl. A01N 55/02; A61K 8/58; A61K 8/92; A61Q 17/00; A61Q 19/10; A01N 25/34; A61K 8/02

(52) U.S. Cl. CPC A01N 55/02; A01N 25/34; A61K 8/58; A61K 8/0216; A61K 8/0254; A61Q 17/005; A61Q 19/10; A61K 8/922; A61K 8/925; A61K 2800/412

(57) ABSTRACT

Antimicrobial bar compositions can include, for example, from about 0.1% to about 35%, by weight of the antimicrobial bar composition, of water; from about 45% to about 99%, by weight of the antimicrobial bar composition, of soap; and from about 0.01% to about 5%, by weight of the antimicrobial bar composition, of platelet zinc pyrithione (“platelet ZPT”). The platelet ZPT includes a median particle diameter of about 2 microns to about 3 microns, a mean particle diameter of about 3 microns to about 4 microns, and a thickness of about 0.6 microns to about 15 microns.

### FIELD OF THE INVENTION

The present invention relates to bar compositions for cleansing skin. More specifically, the present invention relates to antimicrobial bar compositions for cleansing skin comprising platelet zinc pyrithione (“ZPT”).

### BACKGROUND OF THE INVENTION

Human health is impacted by many microbial entities or microbials such as germs, bacteria, fungi, yeasts, molds, viruses, or the like. For example, invasion by microbial entities or microbials including various viruses and bacteria cause a wide variety of sicknesses and ailments. To reduce such an invasion, people frequently wash their skin and, in particular, their hands with antimicrobial bar soaps. Antibacterial bar soaps typically include soaps in combination with, for example, antimicrobial agents. For example, one such antibacterial bar soap is a ZPT bar soap. ZPT bar soaps typically include soap in combination with zinc pyrithione (“ZPT”) in the form of small or fine particles (“particulate ZPT”). When the skin is washed with an antimicrobial bar soap such as a ZPT bar soap, the surfactancy of the soap typically removes most of the microbial entities or microbials on the skin, while the antimicrobial agent such as the particulate ZPT deposits onto the skin to provide residual protection against subsequent invasion.

Unfortunately, current antibacterial soaps such as ZPT bar soaps do not deposit enough antimicrobial agents such as particulate ZPT to effectively protect against, for example, subsequent invasion by the increasing number of microbial entities or microbials on the skin. For example, current ZPT bar soaps do not deposit enough particulate ZPT to prevent subsequent invasion by gram negative bacteria such as E. coli, gram positive bacteria, and the like. Thus, there remains a desire for an antibacterial bar composition that improves the number of microbial entities or microbials removed from the skin and improves the amount of antimicrobial agents or ZPT deposited on the skin.

### SUMMARY OF THE INVENTION

According to one embodiment, the present invention relates to an antimicrobial bar composition comprising: (a) from about 0.1% to about 35%, by weight of the antimicrobial bar composition, of water; (b) from about 45% to about 99%, by weight of the antimicrobial bar composition, of soap; and (c) from about 0.01% to about 5%, by weight of the antimicrobial bar composition, of platelet zinc pyrithione (ZPT), wherein the platelet ZPT comprises a median particle diameter of about 0.5 microns to about 5 microns, a mean particle diameter of about 1 microns to about 4 microns, and a thickness of about 0.6 micros to about 15 microns.

### BRIEF DESCRIPTION OF THE DRAWINGS

FIG. 1 depicts an example image shown via Scanning Electron Microscopy (“SEM”) of platelet ZPT that can be used in an antimicrobial bar composition

FIG. 2 depicts an example image shown via SEM of particulate ZPT that can be used an antimicrobial bar composition.

FIG. 3 depicts a graphical representation of a comparison of the reduction of microbials in a study of an antimicrobial bar composition with ZPT in platelet form (“platelet ZPT”) vs. an antimicrobial bar composition with ZPT in fine particle form (“particulate ZPT”).

FIG. 4 depicts a graphical representation of a comparison of the deposition of ZPT in a study of an antimicrobial bar composition with platelet ZPT vs. an antimicrobial bar composition with particulate ZPT.

### DETAILED DESCRIPTION OF THE INVENTION

While the specification concludes with the claims particularly pointing and distinctly claiming the invention, it is believed that the present invention will be better understood from the following description.

The devices, apparatuses, methods, components, and/or compositions of the present invention can include, consist essentially of, or consist of, the components of the present invention as well as other ingredients described herein. As used herein, “consisting essentially of” means that the devices, apparatuses, methods, components, and/or compositions may include additional ingredients, but only if the additional ingredients do not materially alter the basic and novel characteristics of the claimed devices, apparatuses, methods, components, and/or compositions.

All percentages and ratios used herein are by weight of the total composition and all measurements made are at 25° C., unless otherwise designated.

All measurements used herein are in metric units unless otherwise specified.

The term “bar composition” as used herein, refers to compositions intended for topical application to a surface such as skin or hair to, for example, remove dirt, oil, and the like. The bar compositions of the present invention are rinse-off formulations, in which the product is applied topically to the skin or hair and then is subsequently rinsed within minutes from the skin or hair with water, or otherwise wiped off using an implement such as a puff, washcloth, or the like. According to example embodiments, the bar compositions disclosed herein, refer to conventional solid (i.e. non-liquid) bar soap compositions and mixed soap/synthetic bar soap compositions. Example bar compositions include from about 40% to about 95% of soluble alkali metal soap of C8-C24, preferably C10-C20 fatty acids. Example bar compositions can also include from 0% to 45% of a synthetic anionic surfactant. Preferred bar compositions can be in the form of a solid (e.g., non-flowing) bar soap intended for topical application to skin including milled toilet bars that can be unbuilt (i.e. include less than about 5% of a water-soluble surfactancy builder).

The term “antibacterial cleansing composition” or “antimicrobial cleansing composition” as used herein, refers to a bar composition suitable for application to a surface such as skin or hair to, for example, remove dirt, oil, or the like and to reduce the number of microbials such as germs, bacteria, viruses, or the like from forming on the surface. For example, without wishing to be bound by theory, the antibacterial cleansing compositions or antimicrobial cleansing compositions, herein, can provide protection against subsequent invasions of microbials on the surface by depositing antibacterial agents such as ZPT thereon. The antibacterial cleansing compositions or antimicrobial cleansing compositions, herein, can be effective against Gram positive bacteria, Gram negative bacteria, fungi, yeasts, molds, viruses, or the like.

One embodiment disclosed herein relates to an antimicrobial bar composition that includes from about 45% to about 99% of a soap and from about 0.01% to about 5% of platelet ZPT. The platelet ZPT includes a median particle diameter of about 0.5 microns to about 10, alternatively about 1 to about 5 microns, and alternatively about 3 microns; a mean particle diameter of about 0.5 to about 10 microns, alternatively about 1 to about 5 microns, alternatively about 2 to about 4 microns, and alternatively about 3 microns, and a thickness of about 0.6 to about 15 microns, alternatively about 0.6 to 1 micron, alternatively about 0.6 to about 0.8, and alternatively about 0.6 to about 0.7 microns. The platelet ZPT can also have a span of less than about 5, and alternatively about 1.

Without wishing to be bound by theory, it is believed that the antimicrobial bar compositions of the present invention eliminate problems associated with the formation and/or removal of microbials and/or the deposition of antimicrobials on a surface such as skin and/or hair. Specifically, it has been found that the use of platelet ZPT in antimicrobial bar compositions improve the antimicrobial efficacy on the surface, and, thus, can improve protection against subsequent invasion of microbials on the surface. In particular, the number of microbials that can form on the surface after use of an antimicrobial bar composition comprising platelet ZPT is reduced. Additionally, the efficiency on, for example, a mass basis of the amount of ZPT deposited on the surface after use of the antimicrobial bar composition comprising platelet ZPT is improved. As such, the overall residual efficacy of the antimicrobial bar compositions is also improved resulting in improved protection from subsequent invasions of microbials on the surface.

### Soap

The antimicrobial bar composition of the present invention will typically include from about 40% to about 99.5%, preferably from about 45% to about 75%, and more preferably from about 50% to about 65%, by weight of the composition, of soap. The soap can include a typical soap, i.e., the alkali metal or alkanol ammonium salts of alkane- or alkene monocarboxylic acids. Sodium, magnesium, potassium, calcium, mono-, di- and tri-ethanol ammonium cations, or combinations thereof, are suitable for purposes of the present invention. Generally, the soap included in the antimicrobial bar composition disclosed herein can include sodium soaps or a combination of sodium soaps with from about 1% to about 25% ammonium, potassium, magnesium, calcium or a mixture of these soaps. According to example embodiments, the soaps useful herein are the well known alkali metal salts of alkanoic or alkenoic acids having about 12 to 22 carbon atoms, preferably about 12 to about 18 carbon atoms or alkali metal carboxylates of alkyl or alkene hydrocarbons having about 12 to about 22 carbon atoms.

The antimicrobial bar composition can also include soaps having a fatty acid distribution of coconut oil that can provide the lower end of the broad molecular weight range or a fatty acid distribution of peanut or rapeseed oil, or their hydrogenated derivatives, that can provide the upper end of the broad molecular weight range.

It can be preferred to use soaps in the antimicrobial bar composition that include the fatty acid distribution of tallow and vegetable oil. The tallow can include fatty acid mixtures that typically have an approximate carbon chain length distribution of 2.5% C14, 29% C16, 23% C18, 2% palmitoleic, 41.5% oleic and 3% linoleic. The tallow can also include other mixtures with similar distribution, such as the fatty acids derived from various animal tallows and lard. According to an example embodiment, the tallow can also be hardened (i.e., hydrogenated) to convert part or all of the unsaturated fatty acid moieties to saturated fatty acid moieties.

In an embodiment, the vegetable oil is selected from the group consisting of palm oil, coconut oil, palm kernel oil, palm oil stearine, and hydrogenated rice bran oil, or mixtures thereof, since these are among the more readily available fats with palm oil stearine, palm kernel oil, and/or coconut oil being preferred. According to one embodiment, the coconut oil can include a proportion of fatty acids having at least 12 carbon atoms of about 85%. Such a proportion can be greater when mixtures of coconut oil and fats such as tallow, palm oil, or non-tropical nut oils or fats are used where the principle chain lengths are C16 and higher. According to a preferred embodiment, the soap included in the antimicrobial bar composition can be a sodium soap having a mixture of about 67-68% tallow, about 16-17 coconut oil, and about 2% glycerin, and about 14% water.

According to example embodiments, the soaps included in the antimicrobial bar composition disclosed herein can also include unsaturation in accordance with commercially acceptable standards. For example, in one embodiment, the soaps included in the antimicrobial bar composition disclosed herein can include unsaturation in the ranges of from about 37% to 45% of the saponified material.

In an example embodiment, the soap included in the antimicrobial bar composition can be made by the classic kettle boiling process or modern continuous soap manufacturing processes wherein natural fats and oils such as tallow or coconut oil or their equivalents are saponified with an alkali metal hydroxide using procedures well known to those skilled in the art. Alternatively, the soaps may be made by neutralizing fatty acids such as lauric (C12), myristic (C14), palmitic (C16), or stearic (C18) acids with an alkali metal hydroxide or carbonate.

In a preferred embodiment, the antimicrobial bar composition can include a soap made by a continuous soap manufacturing process. The soap can be processed into soap noodles via a vacuum flash drying process. A preferred soap noodle comprises about 67.2% tallow soap, about 16.8% coconut soap, about 2% glycerin and comprises about 14% water. These percentage amounts are by weight of the soap noodles. The soap noodles are then utilized in a milling process to make the finished antimicrobial bar composition as described below.

### Zinc Pyrithione

According to an example embodiment, the antimicrobial bar composition can further comprise a pyrithione or a polyvalent metal salt of pyrithione such as a zinc salt of 1-hydroxy-2-pyridinethione (known as “zinc pyrithione” or “ZPT”).

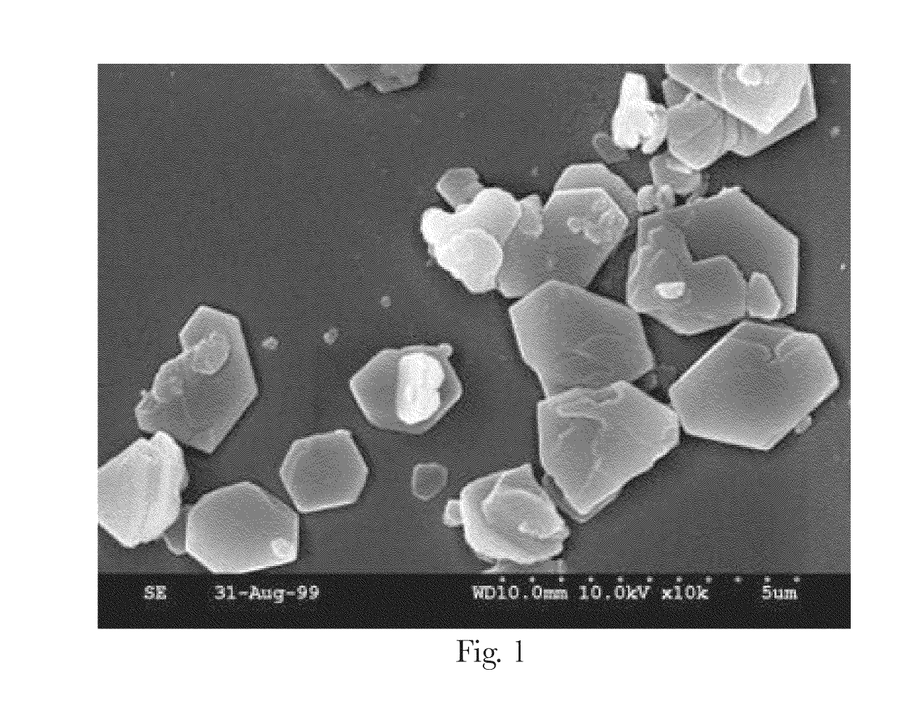

In a preferred embodiment, the zinc pyrithione included in the antimicrobial bar composition is dry powder zinc pyrithione in platelet particle form (“platelet ZPT”). According to example embodiments, the platelet ZPT included in the antimicrobial bar composition can include particles with, for example, a median particle diameter of about 0.5 microns to about 10, alternatively about 1 to about 5 microns, and alternatively about 3 microns and a mean particle diameter of about 0.5 to about 10 microns, alternatively about 1 to about 5 microns, alternatively about 2 to about 4 microns, and alternatively about 3 microns. The platelet ZPT can also have a thickness of about 0.6 to about 15 microns, alternatively about 0.6 to about 1 micron, alternatively about 0.6 microns to about 0.8 microns, and alternatively about 0.6 microns to about 0.7 microns as shown in FIG. 1. The platelet ZPT included in the antimicrobial bar composition can also have a span of less than about 5, and alternatively about 1.

The antimicrobial bar composition can include from about 0.01% to about 5%, by weight of the bar composition, of platelet ZPT, alternatively from about 0.1% to about 2%, and alternatively from about 0.1% to about 1%.

According to an example embodiment, the platelet ZPT can be included in the antimicrobial bar compositions disclosed herein as a dry power that is, for example, dispersed with the soap. Alternatively, the platelet ZPT can be included in the antimicrobial bar compositions disclosed herein as aqueous dispersion with, for example, the soap.

The platelet ZPT included in the antimicrobial bar composition can be stabilized against, for example, flocculation. In one embodiment, each of the platelet ZPTs used in the antimicrobial bar composition can have a coating or layer thereon to prevent the platelet ZPTs from attaching to each other. The coating or layer can be polynaphthalene sulfonate or any other suitable sulfate, sulfonate, carboxylate, or other compound that provides stability for example by charge or steric barrier.

In example embodiments, the ZPT can be made by reacting 1-hydroxy-2-pyridinethione (i.e., pyrithione acid) or a soluble salt thereof with a zinc salt (e.g. zinc sulfate) to form a zinc pyrithione precipitate as illustrated in U.S. Pat. No. 2,809,971 and the zinc pyrithione can be formed or processed into platelet ZPT using, for example, sonic energy as illustrated in U.S. Pat. No. 6,682,724.

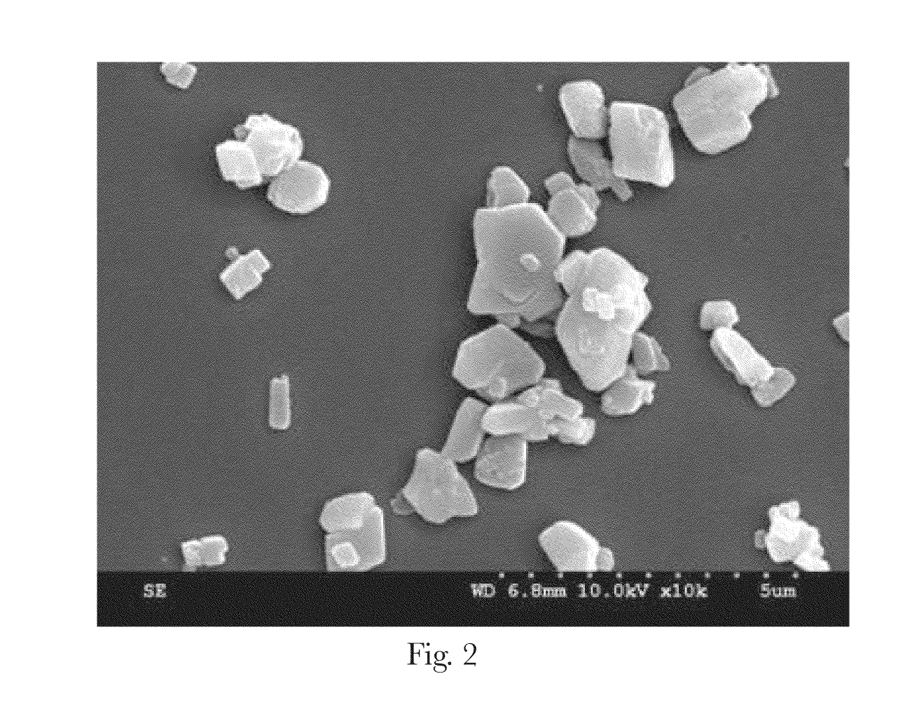

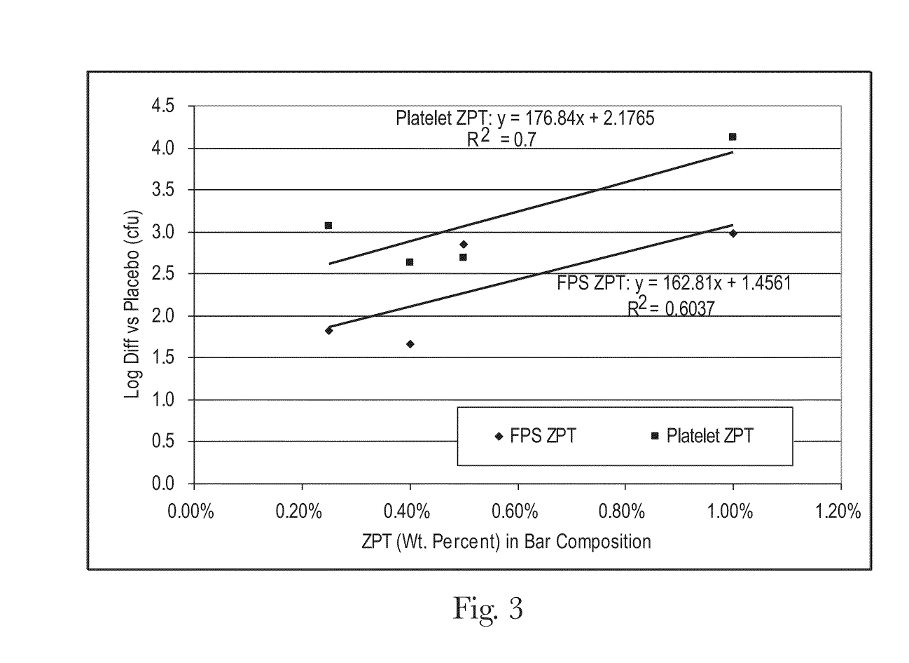

It has been discovered that the use of platelet ZPT in an antimicrobial bar soap such as the antimicrobial bar composition disclosed herein provides improvements in the efficiency of the amount of ZPT deposited on the surface upon which the antimicrobial bar composition is being used on as well as reductions in the amount of antimicrobials that form after use. More specifically, it has been discovered that the use of platelet ZPT having a median particle diameter of about 1 micron to about 5 microns, a mean particle diameter of about 1 microns to about 5 microns, and a thickness of about 0.6 microns to about 15 microns in an antimicrobial bar composition such as the antimicrobial bar composition disclosed herein provides improvements in the efficiency of the amount of ZPT deposited on the surface upon which the antimicrobial bar composition is being used on as well as reductions in the amount of antimicrobials that form after use in comparison with, for example, particulate ZPT such as the particulate ZPT shown in FIG. 2. FIG. 3 illustrates these improvements by comparing an antimicrobial bar composition that includes particulate ZPT having a median particle diameter of about 0.70 microns, a mean particle diameter of about 0.75 microns, and a thickness of less than 0.6 microns with an antimicrobial bar composition that includes platelet ZPT described above. As shown in FIG. 1, the use of platelet ZPT reduces the number of colony forming units (CFUs) that form on a substrate in comparison with particulate ZPT. As such, the use of platelet ZPT increases the residual efficacy of the antimicrobial bar composition and provides protection on the surface the antimicrobial bar composition is used on from subsequent invasions of microbials.

### Water

The antimicrobial bar composition also includes from about 0.1% to about 35%, more preferably from about 0.3% to about 20%, and more preferably about 10%, by weight of the composition, of water.

It should be understood that an amount of water will be lost, i.e. evaporated, during the process of making the antimicrobial bar composition. Also, once the finished product is made, water can be further lost from the antimicrobial bar composition due to water evaporation, water being absorbed by surrounding packaging (e.g. a cardboard carton), and the like.

It can be important to incorporate in the antimicrobial bar composition materials that tend to bind the water such that the water can be maintained in the antimicrobial bar composition. Such materials include carbohydrate structurants, humectants, such as glycerin, as described herein.

### Optional Ingredients

The antimicrobial bar composition can further include various optional ingredients such as structurants, polymers, humectants, fatty acids, inorganic salts, surfactants, other antimicrobial agents or actives, brighteners, silica, and moisturizers or benefit agents as described below.

Hydrophilic Structurants

In one embodiment, the antimicrobial bar composition can optionally include hydrophilic structurants such as carbohydrate structurants, gums, and polymers that tend to assist in maintaining a particular level of water in the antimicrobial bar composition. Suitable structurants as ingredients in the antimicrobial bar composition described herein include carbohydrates such raw starch (corn, rice, potato, wheat, and the like) and pregelatinozed starch; polymers (anionic, nonionic, zwitterionic, or hydrophobically modified) such as carboxymethyl cellulose, stabylene, carbopol, polyethylene glycol, polyethylene oxide; and gums such as carregeenan and xanthan gum.

The level of carbohydrate structurant in the antimicrobial bar composition can be from about 0.1% to about 30%, preferably from about 2% to about 25%, and more preferably from about 4% to about 20%, by weight of the antimicrobial bar composition.

Cationic Polymers

The antimicrobial bar composition can also optionally include cationic polymers to improve the lathering and skin feel benefits of the antimicrobial bar composition during and after use. If present, the antimicrobial bar composition can include from about 0.001% to about 10%, preferably from about 0.01% to about 5%, more preferably from about 0.05% to about 1%, by weight of the composition, of cationic polymer. Preferred embodiments include amounts of cationic polymer of less than about 0.2%, preferably less than about 0.1%, by weight of the composition. If the level of cationic polymer is too high, the resulting antimicrobial composition can exhibit a sticky skin feel.

Suitable cationic polymers for use in the antimicrobial bar composition include, but are not limited to, cationic polysaccharides; cationic copolymers of saccharides and synthetic cationic monomers; cationic polyalkylene imines; cationic ethoxy polyalkylene imines; cationic poly[N-[3-(dimethylammonio)propyl]-N′[3-(ethyleneoxyethylene dimethyl ammonio)propyl]urea dichloride]. Suitable cationic polymers generally include polymers having a quaternary ammonium or substituted ammonium ion.

Suitable cationic polysaccharides encompass those polymers based on 5 or 6 carbon sugars and derivatives which have been made cationic by engrafting of cationic moieties onto the polysaccharide backbone. They can be composed of one type of sugar or of more than one type, i.e. copolymers of the above derivatives and cationic materials. The monomers may be in straight chain or branched chain geometric arrangements. Cationic polysaccharide polymers include: cationic celluloses and hydroxyethylcelluloses; cationic starches and hydroxyalkyl starches; cationic polymers based on arabinose monomers such as those which could be derived from arabinose vegetable gums; cationic polymers derived from xylose polymers found in materials such as wood, straw, cottonseed hulls, and corn cobs; cationic polymers derived from fucose polymers found as a component of cell walls in seaweed; cationic polymers derived from fructose polymers such as Inulin found in certain plants; cationic polymers based on acid-containing sugars such as galacturonic acid and glucuronic acid; cationic polymers based on amine sugars such as galactosamine and glucosamine; cationic polymers based on 5 and 6 membered ring polyalcohols; cationic polymers based on galactose monomers which occur in plant gums and mucilages; cationic polymers based on mannose monomers such as those found in plants, yeasts, and red algae; cationic polymers based on galactommannan copolymer known as guar gum obtained from the endosperm of the guar bean. Non-limiting examples of cationic polysaccharides suitable herein include cationic hydroxyethyl cellulose (available under the tradename Ucare Polymer JR-400®, Ucare Polymer JR-125® or Ucare Polymer LR-400® from Amerchol); cationic starches (available under the tradename STALOK® 100, 200, 300, and 400 from Staley, Inc.); cationic galactomannans based on guar gum (available under the tradename Galactasol® 800 series from Henkel, Inc. and under the tradename JAGUAR® from Meyhall Chemicals, Ltd.).

Suitable cationic copolymers of saccharides and synthetic cationic monomers useful in the antimicrobial bar composition encompass those containing the following saccharides: glucose, galactose, mannose, arabinose, xylose, fucose, fructose, glucosamine, galactosamine, glucuronic acid, galacturonic acid, and 5 or 6 membered ring polyalcohols. Also included are hydroxymethyl, hydroxyethyl and hydroxypropyl derivatives of the above sugars. The synthetic cationic monomers for use in these copolymers can include dimethyidiallylammonium chloride, dimethylaminoethylmethyacrylate, diethyldiallylammonium chloride, N,N-diallyl,N—N-dialklyl ammonium halides, and the like. Non-limiting examples of copolymers of saccharides and synthetic cationic monomers include those composed of cellulose derivatives (e.g. hydroxyethyl cellulose) and N,N-diallyl,N—N-dialkyl ammonium chloride available from National Starch Corporation under the tradename Celquat®.

Humectant

The antimicrobial bar composition can optionally further include one or more humectants. The humectants that can be included in the antimicrobial bar composition are generally selected from the group consisting of polyhydric alcohols, water soluble alkoxylated nonionic polymers, and mixtures thereof and are preferably used at amounts by weight of the composition of from about 0.1% to about 20%, more preferably from about 0.5% to about 15%, and more preferably from about 1% to about 10%.

Humectants such as glycerin can be included antimicrobial bar composition as a result from the production of the soap. For example, glycerin can be a by-product after saponification of the antimicrobial bar composition. The glycerin or at least a portion thereof can be left in the antimicrobial bar composition. Thus, in one embodiment, the humectant can be a component of the soap noodle used in preparation of the antimicrobial bar composition. As a product of the soap reaction, the amount of humectant in the soap noodle is typically no more than about 1%, by weight of the soap noodle.

In one embodiment, it can be advantageous to purposely add additional humectant such as glycerin to the composition. The additional humectant can be added to the soap noodle used in preparation of the present compositions. The additional humectant can be added either before the drying process of the neat soap containing about 30% water, or after the drying process (e.g. into an amalgamator). The total level of humectant in this case will typically be at least about 1%, preferably at least about 2%, more preferably at least about 3%, by weight of the composition. Incorporating additional humectant into the antimicrobial bar composition herein can result in a number of benefits such as improvement in hardness of the antimicrobial bar composition, decreased Water Activity of the antimicrobial bar composition, and lowering the weight loss rate of the antimicrobial bar composition over time due to water evaporation.

Humectants useful for the antimicrobial bar composition herein include glycerin, sorbitol, propylene glycol, butylene glycol, hexylene glycol, ethoxylated glucose, 1, 2-hexane diol, hexanetriol, dipropylene glycol, erythritol, starch, trehalose, diglycerin, xylitol, maltitol, maltose, glucose, fructose, sodium chondroitin sulfate, sodium hyaluronate, sodium adenosin phosphate, sodium lactate, pyrrolidone carbonate, glucosamine, cyclodextrin, salts such as chlorides, sulfates, carbonates, and mixtures thereof.

Water soluble alkoxylated nonionic polymers useful for the antimicrobial bar composition herein include polyethylene glycols and polypropylene glycols having a molecular weight of up to about 1000 such as those with CTFA names PEG-200, PEG-400, PEG-600, PEG-1000, and mixtures thereof.

Free Fatty Acid

The antimicrobial bar composition can also optionally include free fatty acid, typically at an amount of from about 0.01% to about 10%, by weight of the composition. Free fatty acids can be incorporated in the antimicrobial bar composition to provide enhance skin feel benefits, such as softer and smoother feeling skin. Suitable free fatty acids include tallow, coconut, palm and palm kernel fatty acids. A preferred free fatty acid added as an ingredient in the antimicrobial bar composition is palm kernel fatty acid. Other fatty acids can be employed although the low melting point fatty acids, such as lauric acid, can be preferred for ease of processing. Preferred amounts of free fatty acid added to the antimicrobial bar composition are from about 0.5% to about 2%, most preferably from about 0.75% to about 1.5%, by weight of the composition.

Inorganic Salts

The antimicrobial bar composition can optional include inorganic salts. The inorganic can help maintain a particular water content or level (e.g. a Water Activity (“Aw) of an antimicrobial bar composition) of the antimicrobial bar composition and improve hardness of the antimicrobial bar composition. The inorganic salts also help bind the water in the antimicrobial bar composition thereby preventing water loss by evaporation or other means. The antimicrobial bar composition can optionally include from about 0.01% to about 15%, preferably from about 1% to about 12%, and more preferably from about 2.5% to about 10.5%, by weight of the composition, of inorganic salt. Higher levels of inorganic salts are generally preferred. Suitable inorganic salts that can be included in the antimicrobial bar composition include magnesium nitrate, trimagnesium phosphate, calcium chloride, sodium carbonate, sodium aluminum sulfate, disodium phosphate, sodium polymetaphosphate, sodium magnesium succinate, sodium tripolyphosphate, aluminum sulfate, aluminum chloride, aluminum chlorohydrate, aluminum-zirconium trichlorohydrate, aluminum-zirconium trichlorohydrate glycine complex, zinc sulfate, ammonium chloride, ammonium phosphate, calcium acetate, calcium nitrate, calcium phosphate, calcium sulfate, ferric sulfate, magnesium chloride, magnesium sulfate, and the like. In preferred embodiments, the inorganic salts that can be included in the antimicrobial bar composition include sodium tripolyphosphate, magnesium salts (such as magnesium sulfate), and/or tetrasodium pyrophosphate. Magnesium salts, when used as an ingredient in the present antimicrobial bar compositions comprising soap, tend to be converted to magnesium soap in the finished product. Sodium tripolyphosphate, magnesium salts (and as a result magnesium soap), and/or tetrasodium pyrophosphate are preferred in the antimicrobial bar composition. Sodium tripolyphosphate is also preferred as it can tend to promote the generation of lather as the antimicrobial bar composition is used by a consumer for cleansing skin.

Synthetic Surfactant

The antimicrobial bar composition can optionally include synthetic surfactants. Synthetic surfactants useful in the antimicrobial bar composition can further improve the lathering properties of the antimicrobial bar composition during use. The synthetic surfactants useful in the antimicrobial bar composition include anionic, amphoteric, nonionic, zwitterionic, and cationic surfactants. Synthetic surfactants are typically incorporated in the antimicrobial bar composition at an amount of from about 0.1% to about 20%, preferably from about 0.5% to about 10%, and more preferably from about 0.75% to about 5%, by weight of the antimicrobial bar composition.

Examples of anionic surfactants include but are not limited to alkyl sulfates, anionic acyl sarcosinates, methyl acyl taurates, N-acyl glutamates, acyl isethionates, alkyl ether sulfates, alkyl sulfosuccinates, alkyl phosphate esters, ethoxylated alkyl phosphate esters, trideceth sulfates, protein condensates, mixtures of ethoxylated alkyl sulfates and the like. Alkyl chains for such surfactants are C8-22, preferably C10-18 and, more preferably, C12-14 alkyls.

Examples of zwitterionic surfactants which can be used in the antimicrobial bar composition can be exemplified by those which can be broadly described as derivatives of aliphatic quaternary ammonium, phosphonium, and sulfonium compounds, in which the aliphatic radicals can be straight chain or branched and wherein one of the aliphatic substituents contains from about 8 to 18 carbon atoms and one contains an anionic water-solubilizing group, for example, carboxy, sulfonate, sulfate, phosphate, or phosphonate.

Examples of amphoteric surfactants which can be used in the antimicrobial bar composition are those which can be broadly described as derivatives of aliphatic secondary and tertiary amines in which the aliphatic radical can be straight chain or branched and wherein one of the aliphatic substituents contains from about 8 to about 18 carbon atoms and one contains an anionic water solubilizing group, e.g., carboxy, sulfonate, sulfate, phosphate, or phosphonate.

In an example embodiment, the antimicrobial bar composition described herein can includes zwitterionic or amphoteric surfactants such as betaines, amphoacetates, and ethanol-amines.

Examples of suitable cationic surfactants include stearyldimenthylbenzyl ammonium chloride; dodecyltrimethylammonium chloride; nonylbenzylethyldimethyl ammonium nitrate; tetradecylpyridinium bromide; laurylpyridinium chloride; cetylpyridinium chloride; laurylpyridinium chloride; laurylisoquinolium bromide; ditallow(Hydrogenated)dimethyl ammonium chloride; dilauryldimethyl ammonium chloride; and stearalkonium chloride; and other cationic surfactants known in the art.

Nonionic surfactants useful in the antimicrobial bar composition can be broadly defined as compounds produced by the condensation of alkylene oxide groups (hydrophilic in nature) with an organic hydrophobic compound, which may be aliphatic or alkyl aromatic in nature.

A preferred synthetic surfactant for use in the antimicrobial bar composition is sodium laureth-n sulfate (where n is the average number of moles of ethoxylate per molecule and is between 1 and 3). Sodium laureth sulfate tends to provide excellent lathering properties, especially when combined with sodium tripolyphosphate as the inorganic salt in the present compositions.

Antibacterial Agents

The antimicrobial bar composition can optionally further include one or more additional antibacterial agents that can serve to further enhance the antimicrobial effectiveness of the bar compositions. When present, the antimicrobial bar composition can include from about 0.001% to about 2%, preferably from about 0.01% to about 1.5%, more preferably from about 0.1% to about 1%, by weight of the antimicrobial bar composition. Examples of antibacterial agents that can be employed are the carbanilides, for example, triclocarban (also known as trichlorocarbanilide), triclosan, a halogenated diphenylether available as DP-300 from Ciba-Geigy, hexachlorophene, 3,4,5-tribromosalicylanilide, and salts of 2-pyridinethiol-1-oxide, salicylic acid and other organic acids. Other suitable antibacterial agents are described in detail in U.S. Pat. No. 6,488,943 (referred to as antimicrobial actives).

Brighteners

Additionally, brighteners can be included as optional ingredients in the antimicrobial bar composition at an amount of from about 0.001% to about 1%, preferably from about 0.005% to about 0.5%, and more preferably from about 0.01% to about 0.1%, by weight of the composition. Examples of suitable brighteners in the present compositions include disodium4,4′-bis-(2-sulfostyril)-biphenyl (commercially available under the tradename Brightener-49, from Ciba Specialty Chemicals); disodium4,4′-bis-[(4,6-di-anilino-s-triazine-2-yl)-amino]-2,2′-stilbenedisulfonate (commercially available under the tradename Brightener 36, from Ciba Specialty Chemicals); 4,4′-bis-[(4-anilino-6-morpholino-s-triazine-2-yl)-amino]-2,2′-stilbenedisulfonate (commercially available under the tradename Brightener 15, from Ciba Specialty Chemicals); and 4,4′-bis-[(4-anilino-6-bis-2(2-hydrox-yethyl)-amino-s-triazine-2-yl)-amino]-2,2′-stilbenedisulfonate (commercially available under the tradename Brightener 3, from Ciba Specialty Chemicals); and mixtures thereof.

Silica

Silica, or silicon dioxide, can be optionally incorporated in the antimicrobial bar composition at an amount of from about 0.1% to about 15%, preferably from about 1% to about 10%, and more preferably from about 3% to about 7%, by weight of the composition. Silica is available in a variety of different forms include crystalline, amorphous, fumed, precipitated, gel, and colloidal. Preferred forms herein are fumed and/or precipitated silica.

Thickening silica typically has smaller particle size versus normal abrasive silica and is preferred herein. The average particle size of thickening silica is preferably from about 9 μm to about 13 μm, as opposed to normal abrasive silica which has an average particle size of from about 20 μm to about 50 μm. Due to the surface of the preferred thickening silica having a relatively large amount of silinol groups, it can bind the water and build the right texture for the present bar compositions. The silinol groups tend to form hydrogen bonds wherein three-dimensional networks are fabricated to act like a spring in the soap phase to deliver good foaming and good texture. The thickening silica preferably has a high oil absorbency value (DBP), normally indicating porosity and large surface area, and is preferably greater than about 250 (g/100 g), and more preferably greater than about 300 (g/100 g).

Non-limiting examples of suitable thickening silica include: SIDENT 22S commercially available from Degussa; ZEODENT 165 commercially available from J. M. Huber Corp.; SORBOSIL TC15 commercially available from Ineos Silicas; TIXOSIL 43 commercially available from Rhodia; and SYLOX 15X commercially available from W. R. Grace Davidson.

Moisturizers/Emollients

Moisturizers can also optionally be included in the antimicrobial bar composition to provide the skin conditioning benefits and to improve the mildness of the product. The selection of the levels and types of moisturizers to be incorporated into the product is made without adversely affecting the stability of the product or its in-use characteristics, thereby delivering good moisturization and lather.

Both occlusive and nonocclusive moisturizers are suitable for use in the present invention. Some examples of moisturizers are long chain fatty acids, liquid water-soluble polyols, glycerin, propylene glycol, sorbitol, polyethylene glycol, ethoxylated/propoxylated ethers of methyl glucose (e.g., methyl gluceth-20) and lanolin alcohol (e.g., Solulan-75).

When moisturizers are used in the compositions of the present invention they are used at levels of from about 2% to about 20% by weight of the composition. The preferred and more preferred levels of moisturizers are, respectively, 4% to 15% and 8% to 12%. The preferred moisturizers are the coconut and tallow fatty acids. Some other preferred moisturizers are the nonocclusive liquid water-soluble polyols (e.g., glycerin) and the essential amino acid compounds found naturally in the skin.

Other preferred nonocclusive moisturizers are compounds found to be naturally occurring in the stratum corneum of the skin, such as sodium pyrrolidone carboxylic acid, lactic acid, urea, L-proline, guanidine and pyrrolidone. Examples of other nonocclusive moisturizers include hexadecyl, myristyl, isodecyl or isopropyl esters of adipic, lactic, oleic, stearic, isostearic, myristic or linoleic acids, as well as many of their corresponding alcohol esters (sodium isostearoyl-2-lactylate, sodium capryl lactylate), hydrolyzed protein and other collagen-derived proteins, aloe vera gel and acetamide MEA (acetmonoethanolamide).

Other optional ingredients in the antimicrobial bar composition include: perfumes; sequestering agents, such as tetrasodium ethylenediaminetetraacetate (EDTA), EHDP or mixtures thereof typically in an amount of 0.01 to 1%, preferably 0.01 to 0.05%, by weight of the composition; and coloring agents, opacifiers and pearlizers such as titanium dioxide; all of which are useful in enhancing the appearance or cosmetic properties of the product.

The pH of a 10% solution of, for example, the antimicrobial bar composition dissolved in water can be greater than about 10, alternatively greater than about 10.7. According to an example embodiment, the pH of the antimicrobial bar soap disclosed herein can be measured using any commercially available pH meter at about 25° C.

Additionally, the present bar compositions will preferably exhibit a Water Activity (“Aw”) of less than about 0.92, alternatively less than about 0.9, alternatively less than about 0.85, and alternatively less than about 0.80, as measured by the “Water Activity Test Method” described herein.

The appearance of the antimicrobial bar composition according to the present invention can be transparent, translucent, or opaque. In one embodiment, the antimicrobial bar composition is opaque.

According to example embodiments, the antimicrobial bar compositions of the present invention can be used by consumers to cleanse skin during bathing or washing.

### Process of Manufacture

The bar composition of the present invention can be made via a number of different processes known in the art. Preferably, the present compositions are made via a milling process, resulting in milled bar compositions.

A typical milling process of manufacturing a bar composition includes: (a) a crutching step in which the soap is made, (b) a vacuum drying step in which the soap is made into soap noodles, (c) an amalgamating step in which the soap noodles are combined with other ingredients of the bar composition, (d) a milling step in which a relatively homogeneous mixture is obtained, (e) a plodding step in which the soap mixture is extruded as soap logs and then cut into soap plugs, and (f) a stamping step in which the soap plugs are stamped to yield the finished bar soap composition.

### Test/Study Methods

Pigskin Kill Rate Test/Study

1. Preparation of Placebo (E. coli Cell Culture)

To prepare the placebo, perform a one wash/rinse performance protocol. In particular, generate an overnight bacterial culture of E. coli (strain 10536, 8879, or 11259) by inoculating 50 ml of TSB with one colony obtained from a Tryptic Soy Agar (TSA) streak plate. Grow the culture for 17-18 hr, 37° C., 200 rpm in a dry shaker.

2. Kill Rate Test

To determine efficacy of a bar soap, perform bar soap ex vivo performance tests on pigskins. First, obtain, clean, refrigerate, and irradiate (25-40 kGy) the pigskins. Store the irradiated pigskins at −20° C. until testing. To test the bar soap compositions, thaw 10×10 cm pigskins to room temperature for 1 hr, and cut the pigskins into 5×10 cm sections using a sterile scalpel.

Using a gloved hand, wash the pigskins as follows: Rinse a 5×10 cm pigskin for 15 sec, with tap water at 33-36° C. with a flow rate of 4-4.2 L/min. Wet the bar soap composition in the running water for 5 sec, lay the bar composition flat on the pigskin surface, then immediately rub the bar soap composition gently across the entire pigskin surface for 15 sec using back and forth motions and light hand pressure similar to that during conventional hand washing. Then, generate lather by continuously rubbing the pigskin for 45 seconds with the hand (e.g. absent the bar soap composition). Rinse the pigskin with tap water for 15 sec by holding the tissue at a 45 degree angle and allowing the water to impinge on the top surface and cascade downwards across the entire surface. Lightly pat the pigskin dry with a sterile tissue, and allow the pigskin to dry for 5-10 min in still room air under low light conditions.

Cut the pigskin into 2×2.5 cm slices and inoculate each slice with 10⁶-10⁷ cfus by using 10 ul of a 1:20 dilution of Tryptic Soy Broth (TSB) obtained from an overnight culture as described above. Allow the bacteria to dry on the slice of the pigskin surface for 20 min, then place the slice of the pigskin into a humidified chamber (60% RH, 33° C.), and incubate the slices for 0 h, 2 h, or 5 h. After incubation, place the slice into a jar containing 50 ml of ice cold neutralization buffer of Modified Leethen Broth with 1.5% Tween-80 and 1% Lecithin (MBL-T), and vigorously shake the buffer with the slice therein for 1 min to elute bacteria. As necessary, dilute the suspension in MBL-T and place the suspension onto Tryptic Soy Agar (TSA) plates to obtain cell counts. Incubate the plates for 24 h, at 33° C., and 60% Relative Humidity. Then, count the TSA plates (e.g. the cfus thereof) to calculate the cfu/ml and generate a growth curve using GraphPad Prism v4.1. Perform the pigskin kill rate test/study described above once to calculate the cfu/ml and to generate the growth curve. (Note: The pigskin kill rate/set described above can also be performed multiple times and the data for each repetition can be averaged (e.g. each of the calculated cfu/ml for each repetition can be averaged together.)

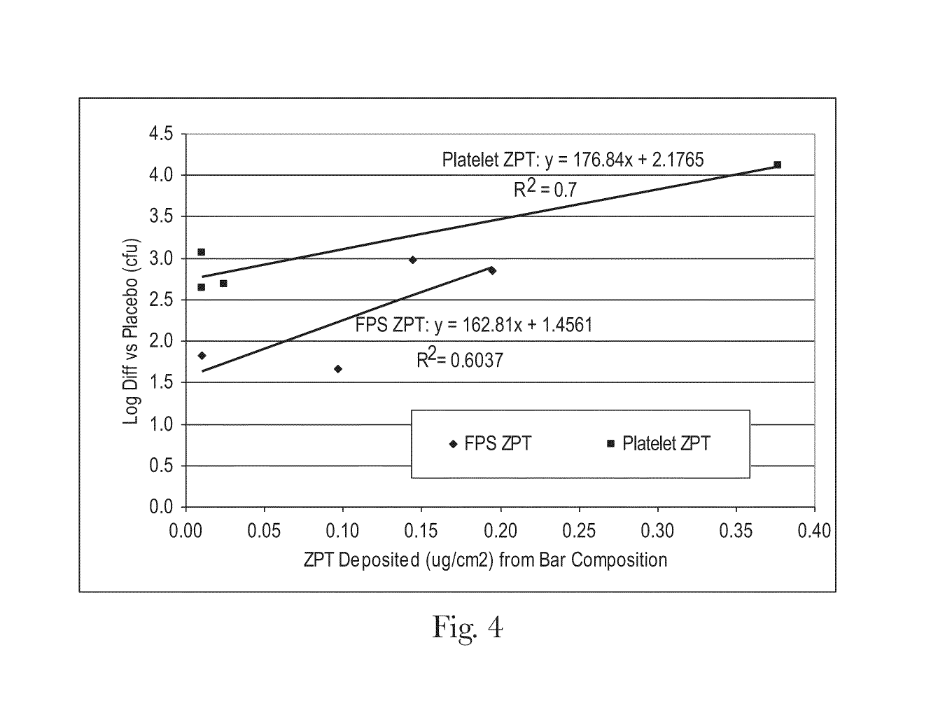

The cfu/ml and growth curves based thereon such as those shown in Table 2 below and FIGS. 3-4 can be calculated using, e.g., the delta between the control or sample and the placebo. For example, the differences can be calculated using the log values of the placebo at the starting time of (e.g. t=0 h) of the pigskin kill rate test and the ending time of (e.g. t=5 h) of the pigskin kill rate test. Specifically, the difference can be calculated according to the following: [(placebo log CFU t5)−(placebo log CFU t0)]+[(sample log CFU t0)−(sample log CFU t5)] (generally referred to as (log CFU growth on placebo)+(log CFU kill for a given sample)). Based on the foregoing calculation, a positive value associated with a sample corresponds to an efficacy or kill rate of cfus associated with that sample (verses, e.g., the placebo) whereas a negative value corresponds to a growth of cfus (such as that shown in Table 2 for the placebo). Additionally, the larger the positive value, the greater the efficacy or kill rate is of a particular sample. (Note: The cfu/ml and growth curves can be offset based on the staring value of the placebo and also can be averaged together if multiple repetitions of the kill test/study are performed as discussed above.)

Particle Size Test Method

ZPT particle size can be measured by conventional light scattering means, such as a Horiba LA-910 particle size analyzer with flow cell. More specifically, disperse a ZPT suspension in water to the target optical density, about 90% and measure the particle sizes with, for example, the Horiba LA-910 particle size analyzer, which uses spherical assumptions for all calculations and calculates the particle size and other parameters based on volume distribution. A relative refractive index of 1.28 with no imaginary portion is used for the calculations and agitation set on 2. The span is a unitless parameter calculated as the breadth of the distribution as [D90−D10]/D50 using the mean diameters at 90%, 10% and 50% of the distribution.

Deposition Test Method

To determine the amount of ZPT deposited on a substrate, perform a cup scrub procedure. To perform the cup scrub procedure, apply an extraction solvent or solution such as an extraction solvent comprised of 80% 0.05M EDTA and 20% ethanol to a substrate surface such as the 2×2.5 cm rectangular pieces of pigskin discussed above to solubilize and remove the ZPT (platelet and particulate). For example, place a 2 cm diameter glass cup that includes 1 ml of extraction solution on the substrate surface. Agitate or rub the substrate area circumscribed by the glass cup and in contact with the extraction solution with a glace policeman for 30 seconds. After agitation or rubbing, remove the extraction solution from the glass cup via a transfer pipette and place the first aliquot of extraction solution in an amber glass vial. Repeat the procedure, e.g., place a 2 cm diameter glass cup that includes 1 ml a second aliquot of extraction solution, agitate or rub as indicated above, and remove the second aliquot solution from the glass cup via a transfer pipette. Then, add the second aliquot of extraction solution to the amber glass vial that includes the first aliquot of extraction solution (a total of 2 ml of extraction solution per extracted area). Then, analyze the extraction solution (combined first and second aliquots) using a HPLC-UV measurement such that a measure of ZPT per unit volume of extraction solution can be yielded. Next, calculate ZPT per deposited per unit area based on the ZPT per unit volume and the surface area of the extracted region of the substrate surface.

Water Activity Test Method

Water Activity (“Aw”) is a measurement of the energy status of the water in a composition. Water activity (“Aw”) is defined as the ratio of the water vapor pressure over a sample (P) to pure water vapor pressure at the same temperature (P₀), expressed fractionally:

Aw=P/P₀

Water activity is measured by a number of conventional, automated techniques including but not limited to the chilled-mirror dewpoint, and capacitance of the equilibrium headspace over a composition. At equilibrium, the relative humidity of the air in the chamber is the same as the water activity of the sample.

For purposes of the present invention, the Aw of a bar composition can be measured using the AquaLab Series 3 Water Activity Meter available from Decagon Devices, Inc. of Pullman, Wash. USA. The Water Activity is measured at 25° C. utilizing the following procedure:
1. Check the sample container of the meter to make sure it is clean and dry before the test;
2. Cut a bar soap composition into 0.2 to 0.4 cm thick pieces with stainless knife;
3. Put pieces into the container of the meter to a ⅓″ to ½″ depth;
4. Press the composition with a gloved finger lightly to make sure the bottom of the container is covered;
5. Put the sample container back into the sample cabinet of the meter and cover it, and turn dial to activate the meter;
6. Wait for the equilibrium until a green LED flashing and/or beeps; and
7. Record the test temperature and Aw.

### EXAMPLES

The following examples describe and demonstrate embodiments within the scope of the invention. The examples are given solely for the purpose of illustration and are not to be construed as limitations of the present invention, as many variations thereof are possible without departing from the spirit and scope of the invention.

### Antimicrobial Compositions and Comparisons

In these examples, the Soap Noodles are made via a conventional process involving a crutching step and a vacuum drying step. The Soap Noodles are then added to an amalgamator. The ingredients of water and platelet ZPT are added to the amalgamator and then mixed for about 30 to 45 seconds. This soap mixture is then processed through conventional milling, plodding, and stamping steps to yield finished bar compositions. According to example embodiments, the finished bar composition can be similar to exiting bar soaps or may be slightly smaller (e.g. can have dimensions half of a typical bar soap). For example, the finished bar composition can be approximately 60-120 grams in weight and can have a bar shape that is rectangular, oval, circulate, or the like with flat surfaces on each side or one or more curved surfaces on each side.

TABLE 1

| | Inventive | Inventive | Inventive | Inventive | Comparative | Comparative | Comparative | Comparative |
|---|---|---|---|---|---|---|---|---|
| Ingredient | Ex. 1 | Ex. 2 | Ex. 3 | Ex. 4 | Ex. 1 | Ex. 2 | Ex. 3 | Ex. 4 |
| Soap | 98.38% | 97.78% | 97.38% | 95.38% | 98.86% | 98.55% | 98.34% | 97.30% |
| Noodle ^{a} | | | | | | | | |
| Platelet | 0.25% | 0.4% | 0.5% | 1.0% | — | — | — | — |
| ZPT ^{b} | | | | | | | | |
| Particulate | — | — | — | — | 0.25% | 0.4% | 0.5% | 1.0% |
| ZPT ^{c} | | | | | | | | |
| Brightener- | 0.02% | 0.02% | 0.02% | 0.02% | 0.02% | 0.02% | 0.02% | 0.02% |
| 49 | | | | | | | | |
| TiO₂ | 0.50% | 0.50% | 0.50% | 0.50% | 0.50% | 0.50% | 0.50% | 0.50% |
| Perfume | 1.10% | 1.10% | 1.10% | 1.10% | 1.10% | 1.10% | 1.10% | 1.10% |
| Water | QS | QS | QS | QS | QS | QS | QS | QS |
| Moisture | −1.00% | −1.00% | −1.00% | −1.00% | −1.00% | −1.00% | −1.00% | −1.00% |
| Loss | | | | | | | | |

^{a} The Soap Noodle utilized in these examples has the following approximate composition: about 67.2% Tallow Soap, about 16.8% Coconut Soap, about 2% Glycerin and about 14% water. These percentage amounts are by weight of the Soap Noodle.
^{b} U2 Zinc Pyrithione, added from 25% active suspension, Arch Chemicals, Inc., Norwalk, Connecticut, USA
^{c} Fine Particle Size Zinc Pyrithione, added from 48% active suspension, Arch Chemicals, Inc.

TABLE 2

| | | | | | Starting | ZPT | Placebo |
|---|---|---|---|---|---|---|---|
| | | Weight | log cfu | log cfu | Count log | deposition | Growth log |
| Example | ZPT | Percent ZPT | (start) | (end) | difference | (ug/cm\hat{ }2) | difference |
| Placebo | n/a | n/a | 4.6 | 5.9 | −1.3 | n/a | n/a |
| Comparative | FPS | 0.25% | 4.8 | 4.3 | 0.5 | 0.01 | 1.8 |
| Ex. 1 | | | | | | | |
| Comparative | FPS | 0.40% | 4.6 | 4.2 | 0.4 | 0.10 | 1.7 |
| Ex. 2 | | | | | | | |
| Comparative | FPS | 0.50% | 4.7 | 3.1 | 1.5 | 0.19 | 2.9 |
| Ex. 3 | | | | | | | |
| Comparative | FPS | 1.00% | 4.7 | 3.0 | 1.7 | 0.14 | 3.0 |
| Ex. 4 | | | | | | | |
| Inventive | Platelet | 0.25% | 4.8 | 3.0 | 1.8 | 0.01 | 3.1 |
| Ex. 1 | | | | | | | |
| Inventive | Platelet | 0.40% | 4.7 | 3.4 | 1.3 | 0.01 | 2.6 |
| Ex. 2 | | | | | | | |
| Inventive | Platelet | 0.50% | 4.7 | 3.4 | 1.4 | 0.02 | 2.7 |
| Ex. 3 | | | | | | | |
| Inventive | Platelet | 1.00% | 4.8 | 2.0 | 2.8 | 0.38 | 4.1 |
| Ex. 4 | | | | | | | |

FIGS. 3-4 and Table 2 illustrate, respectively, a comparison of microbial reduction and deposition of ZPT in a study of inventive examples 1-4 vs. comparative examples 1-4. As shown in FIG. 3 and Table 2, inventive examples 1-4 showed an overall improvement in the number of microbials (e.g. colony forming units (cfus)) that are reduced or formed in comparison to a placebo after use of a bar composition such as the bar composition described herein that includes platelet ZPT vs. a bar composition that includes particulate ZPT (e.g. fine particle ZPT). Furthermore, as shown in FIG. 4 and Table 2, inventive examples 1-4 showed an overall improvement in the efficiency of the amount of ZPT deposited.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An antimicrobial bar composition comprising:
(a) from about 0.1% to about 35%, by weight of the antimicrobial bar composition, of water;
(b) from about 45% to about 99%, by weight of the antimicrobial bar composition, of soap;
(c) from about 0.01% to about 5%, by weight of the antimicrobial bar composition, of platelet zinc pyrithione (platelet ZPT), wherein the platelet ZPT comprises a mean particle diameter of about 0.5 microns to about 10 microns, a median particle diameter of about 0.5 microns to about 10 microns, and a thickness of about 0.6 microns to about 15 microns.

2. The antimicrobial bar composition of claim 1, wherein the mean particle diameter of the platelet ZPT is about 2 microns to about 4 microns.

3. The antimicrobial bar composition of claim 1, wherein the median particle diameter of the platelet ZPT is about 1 micron to about 5 microns.

4. The antimicrobial bar composition of claim 1, wherein the median particle diameter of the platelet ZPT is about 0.6 microns to about 0.7 microns.

5. The antimicrobial bar composition of claim 1, wherein the antimicrobial bar composition comprises from about 0.1% to about 1.0%, by weight of the antimicrobial bar composition, of the platelet ZPT.

6. The antimicrobial bar composition of claim 1, wherein the antimicrobial bar composition comprises from about 40% to about 90%, by weight of said composition, of the soap.

7. The antimicrobial bar composition of claim 6, wherein the soap comprises soaps comprising coconut, tallow, palm or palm kernel fatty acid.

8. The antimicrobial bar composition of claim 1, wherein said composition further comprises an additional antibacterial agent.

9. The antimicrobial bar composition of claim 8, wherein the additional antibacterial agent is selected from the group consisting of triclocarban; triclosan; a halogenated diphenylether; hexachlorophene; 3,4,5-tribromosalicylanilide; salts of 2-pyridinethiol-1-oxide; and mixtures thereof.

10. The antimicrobial bar composition of claim 1, wherein the antimicrobial bar composition comprises about 0.25% to about 1%, by weight of the antimicrobial bar composition, of the platelet ZPT.

11. The antimicrobial bar composition of claim 10, wherein the antimicrobial bar composition comprises a log reduction of colony forming units (cfus) from a placebo (“a placebo log reduction”) of about 2.6 or greater at about 0.25% to about 1%, by weight of the antimicrobial bar composition, of the platelet ZPT.

12. The antimicrobial bar composition of claim 1, wherein said composition comprises a water activity (Aw) of about 0.92 or less.

13. The antimicrobial bar composition of claim 1, wherein the platelet ZPT comprises a span of about 5 or less.

14. An antimicrobial bar composition comprising:
(a) from about 0.1% to about 35%, by weight of the antimicrobial bar composition, of water;
(b) from about 45% to about 99%, by weight of the antimicrobial bar composition, of soap;
(c) from about 0.01% to about 1%, by weight of the antimicrobial bar composition, of platelet zinc pyrithione (platelet ZPT), wherein the platelet ZPT comprises a mean particle diameter of about 2 microns to about 4 microns, a median particle diameter of about 1 microns to about 5 microns, and a thickness of about 0.6 microns to about 0.8 microns.

15. The antimicrobial bar composition of claim 14, wherein the antimicrobial bar composition comprises about 0.25% to about 1%, by weight of the antimicrobial bar composition, of the platelet ZPT.

16. The antimicrobial bar composition of claim 15, wherein the antimicrobial bar composition comprises a log reduction of colony forming units (cfus) from a placebo (“a placebo log reduction”) of about 2.6 or greater at about 0.25% to about 1%, by weight of the antimicrobial bar composition, of the platelet ZPT.

17. The antimicrobial bar composition of claim 14, wherein said composition comprises a water activity (Aw) of about 0.92 or less.

18. The antimicrobial bar composition of claim 14, wherein the platelet ZPT comprises a span of about 1 or less.

19. The antimicrobial bar composition of claim 14, wherein the soap comprises soaps comprising coconut, tallow, palm or palm kernel fatty acid.

20. The antimicrobial bar composition of claim 14, wherein said composition further comprises an additional antibacterial agent, and wherein the additional antibacterial agent is selected from the group consisting of triclocarban; triclosan; a halogenated diphenylether; hexachlorophene; 3,4,5-tribromosalicylanilide; salts of 2-pyridinethiol-1-oxide; and mixtures thereof.
